(19) United States
(12) Patent Application Publication
(10) Pub. No.: US 2008/0213376 A1
(43) Pub. Date: Sep. 4, 2008

(54) Medicament that is Intended for Oral Administration, Comprising a Cyclooxygenase-2 Inhibitor, and Preparation Method Thereof

(75) Inventors: Marinette Moreau, Saint Germain (FR); Nicolas Osty, Luxeuil (FR)

Correspondence Address: OBLON, SPIVAK, MCCLELLAND MAIER & NEUSTADT, P.C., 1940 DUKE STREET, ALEXANDRIA, VA, 22314, US

(73) Assignee: VETOQUINOL, LURE CEDEX (FR)

(21) Appl. No.: 11/814,626

(22) PCT Filed: Jan. 23, 2006
(86) PCT No.: PCT/FR06/00144

§ 371 Date: Feb. 21, 2008

(30) Foreign Application Priority Data
Jan. 24, 2005 (FR) 0500708

(51) Int. Cl. A61K 9/16; A61K 31/4164

(52) U.S. Cl. 424/489; 514/385

(57) ABSTRACT

The invention relates to a medicament which is intended for oral administration, which comprises a cyclooxygenase-2 inhibitor and which has improved bioavailability, and to a method of preparing said medicament. The inventive medicament comprises an agglomerate based on inert solid particles based on at least one excipient, said agglomerate comprising a cyclooxygenase-2 inhibitor and at least one hydrophilic polymer. According to the invention, the agglomerate comprises a spray which is applied to the aforementioned particles, consisting of a solution or suspension of micronized grains of the inhibitor in said polymer(s), in order to agglomerate said particles. The inventive method essentially comprises the following steps, namely: (i) the preparation of a sprayable liquid that is based on the micronized grains of said inhibitor in solution or in suspension in at least one hydrophilic polymer; and (ii) the spraying of the liquid onto the solid particles, in order to obtain the agglomerate by means of wet granulation, said agglomerate comprising the grain solution or suspension spray.

The present invention relates to a medicament that is intended for oral administration, which comprises a cyclooxygenase-2 inhibitor and which has an improved bioavailability, and to a process for preparing this medicament.

In a known manner, many active principles used in medicaments that are administered orally, such as anti-inflammatories, have the drawback of being formed from solid particles that are not very soluble in aqueous media, which adversely affects the oral bioavailability of these medicaments.

On that subject, mention may especially be made of the active principles having a therapeutic and/or prophylactic anti-inflammatory effect belonging to the family of cyclooxygenase-2 inhibitors including, in a nonlimiting manner, a large number of substituted pyrazolyl benzenesulfonamides, such as celecoxib and deracoxib (see U.S. Pat. No. 5,466,823), substituted isoxazolyl benzenesulfonamides, such as valdecoxib (see U.S. Pat. No. 5,633,272), (methylsulfonyl)phenyl furanones, such as rofecoxib (see U.S. Pat. No. 5,474,995 and U.S. Pat. No. 5,981,576), substituted pyridines, such as etoricoxib (see U.S. Pat. No. 5,861,419), 2-(3,5-difluorophenyl)-3-[4-(methylsulfonyl)phenyl]-2-cyclopentene-1-one (see document EP-A-863 134), benzopyranes (see U.S. Pat. No. 6,034,256), substituted pyridazinones (see document WO-A-00/24719) and imidazoles such as cimicoxib (see document EP-B-1 122 243).

Document EP-B-1 122 243 presents, on page 11, a medicament that is intended for oral administration, for example in the form of a tablet, which comprises in its core a cyclooxygenase-2 inhibitor of imidazole type mixed with an inert diluent, a binder and a lubricant, and an outer film provided to delay the disintegration and absorption of the medicament until the gastro-intestinal area of the body. This outer film may be based on sugar, gelatin, hydroxypropyl cellulose or an acrylic resin.

Given that the absorption of these active principles in the digestive tract is limited, the therapeutic dose to be administered must be increased to overcome this drawback. This is the reason why recently it has been sought to improve the bioavailability of these inhibitors for one and the same administration dose. One of the simplest ways of improving the bioavailability is to increase the solubility of the active principle. This parameter may be modified in various ways, by addition of solubilizing agents, surfactants, cyclodextrin, hydrophilic polymers, or else by modifying the structure of the inhibitor particles and by using solid dispersion techniques.

Document WO-A-03/030876 presents a medicament that is intended for oral administration in the form of a tablet that disintegrates in the mouth, which comprises an aqueous dispersion of valdecoxib grains by way of cyclooxygenase-2 inhibitor, these grains being mixed with one or more excipients such as saccharides which are present in the majority in the medicament, to obtain a liquid that is dried by spray drying.

A major drawback of these orally administered medicaments that comprise a cyclooxygenase-2 inhibitor lies especially in their bioavailability that is relatively unsatisfactory and varies from one individual to another.

One object of the present invention is to overcome this drawback, and this object is achieved as the Applicant has just surprisingly discovered that spraying inert solid particles based on at least one excipient with a solution or suspension of micronized grains of a cyclooxygenase-2 specific inhibitor in at least one hydrophilic polymer makes it possible to obtain a medicament that is intended for oral administration and that has an improved bioavailability, in comparison with that of medicaments of the prior art, that incorporate a cyclooxygenase-2 inhibitor, such as cimicoxib, this medicament according to the invention comprising an agglomerate of said solid particles that are agglomerated by the product of spraying this solution or suspension.

According to the invention, this cyclooxygenase-2 specific inhibitor is composed of at least one compound as described in the aforementioned Patent document EP-B-1 122 243 and preferably corresponding to the formula (I) below, or else to that of a salt or solvate of this compound:

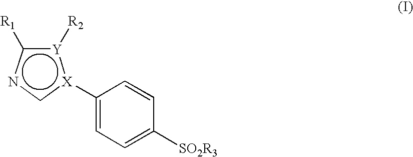

where:
one of the components X and Y represents N and the other represents C;
R₁ represents a hydrogen, methyl, halogen, cyano, nitro, —CHO, —COCH₃ or —COOR₄ group;
R₂ represents an aryl or heteroaryl group optionally substituted by one or more groups chosen independently from halogen, C₁₋₈ alkyl, C₁₋₈ haloalkyl, R₄OC₀₋₈ alkyl, R₄SC₀₋₈ alkyl, cyano, nitro, —NR₄R₆, —NR₄SO₂R₅, —SOR₅, —SO₂R₅, —SO₂NR₄R₆, or —CONR₄R₆ groups;
R₃ represents a C₁₋₈ alkyl, C₁₋₈ haloalkyl or —NR₄R₆ group;
R₄ represents a hydrogen, C₁₋₈ alkyl or C₀₋₈ alkyl aryl group (where the aryl group may optionally be substituted by one or more groups chosen from C₁₋₈ alkyl, halogen, C₁₋₈ haloalkyl, cyano, nitro, R₇OC₀₋₈ alkyl, R₇SC₀₋₈ alkyl, —NR₇R₈, —NR₇COR₅, —COR₇ or —COOR₇ groups);
R₅ represents a C₁₋₈ alkyl or C₁₋₈ haloalkyl group;
R₆ represents a hydrogen, C₁₋₈ alkyl, aryl C₁₋₈ alkyl (where the aryl group may optionally be substituted by one or more groups chosen from C₁₋₈ alkyl, halogen, C₁₋₈ haloalkyl, cyano, nitro, R₇OC₁₋₈ alkyl, R₇SC₀₋₈ alkyl, —NR₇R₈, —NR₇COR₅, —COR₇ or —COOR₇ groups), —COR₈ or —COOR₈ group;
R₇ represents a hydrogen, C₁₋₈ alkyl or benzyl group;
R₈ represents a C₁₋₈ alkyl or C₁₋₈ haloalkyl group;
the aryl group in the definitions above represents a phenyl or naphthyl group; and
the heteroaryl group in the definitions above represents a pyridine, pyrazine, pyrimidine or pyridazine group, which may optionally be fused to a benzene ring.

Even more preferably, by way of cyclooxygenase-2 inhibitor, at least one imidazole, such as cimicoxib, is used that corresponds in a known manner to the formula (II) below:

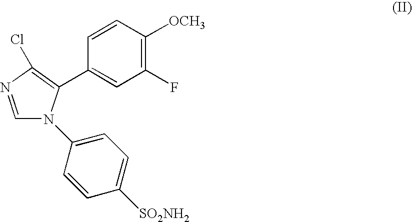

According to one advantageous feature of the invention, said solid particles of excipient(s) are soluble or dispersible in an aqueous medium. Generally, these particles of excipient(s) are hydrophilic, possibly being of crystalline or amorphous structure.

Preferably, by way of excipient(s), water-soluble or water-dispersible particles are used which are chosen from the group consisting of sugars, preferably lactose or saccharose, starch hydrolysates such as malto-dextrin, microcrystalline cellulose, sorbitols and mixtures of several of these compounds.

Even more preferably, said solid particles comprise, in addition, at least one acid that is mixed with said excipient(s), such as, preferably, citric acid, or else tartaric acid or fumaric acid, which makes it possible to increase the solubility of said inhibitor in the body.

According to one main feature of the invention, said agglomerate is capable of being obtained by wet granulation in a device, such as a fluidized air bed.

Preferably, said agglomerate according to the invention specifically comprises the product of spraying said solid particles with a solution of said inhibitor in said hydrophilic polymer(s). Indeed, the Applicant has been able to prove that, unexpectedly, dissolving said inhibitor in this or these polymer or polymers gives the medicament according to the invention a bioavailability that is further improved in comparison with that given by suspending the same inhibitor in the same hydrophilic polymer(s).

These micronized grains of said inhibitor are preferably such that around 90% of them have a largest dimension in cross section which is less than 20 μm.

Preferably, said or at least one of said hydrophilic polymer(s) is chosen from the group consisting of polyvinylpyrrolidones, polyethylene glycols or macrogols, polyvinyl alcohols, cellulose polymers such as hydroxypropylmethyl cellulose, hydroxypropyl cellulose, hydroxymethyl cellulose and carboxymethyl cellulose, methacrylic copolymers, starch, dextrins, gelatin and blends of several of these polymers.

Even more preferably, said or at least one of said hydrophilic polymer(s) is chosen from the group consisting of polyvinylpyrrolidones and polyethylene glycols or macrogols.

Even more preferably still, at least one polyethylene glycol or macrogol is used having a weight-average molecular weight M_{w} ranging from 190 to 9000 g/mol and, even more preferably, ranging from 250 to 600 g/mol and advantageously from 285 to 420 g/mol.

According to one particularly advantageous embodiment of the invention, as hydrophilic polymers, a blend of said polyethylene glycol or macrogol and a polyvinylpyrrolidone having a weight-average molecular weight M_{w} ranging from 2000 to 1 000 000 g/mol, preferably ranging from 5000 to 55 000 g/mol, is used.

According to another advantageous feature of the invention, said product of spraying the solution or suspension of said inhibitor in said polymer(s) comprises, in addition, at least one amphoteric, ionic (i.e. anionic or cationic) or nonionic surfactant.

As a surfactant that can be used, mention may, for example, be made nonlimitingly of:
sodium lauryl sulfate;
polyethoxylated sorbitan esters, or polysorbates; and
poloxamers.

It is also possible to use mixtures of several of these surfactants. Preferably, sodium lauryl sulfate is used as the surfactant.

According to another advantageous feature of the invention, said agglomerate comprises said inhibitor according to a weight fraction ranging from 1% to 20% and, preferably, ranging from 3% to 10%.

Advantageously, said agglomerate comprises said excipient(s) according to a weight fraction ranging from 10% to 80% and, preferably, ranging from 30% to 75%.

Also advantageously, said agglomerate comprises said hydrophilic polymer(s) according to a weight fraction ranging from 3% to 30% and, preferably, ranging from 12% to 25%.

Also advantageously, the weight fraction of said surfactant(s) in said agglomerate varies from 0.1% to 6%.

According to another feature of the invention, said medicament may optionally comprise at least one outer layer covering said particle agglomerate and comprising compatible additives chosen from the group consisting of disintegrating agents, fillers, pigments, flavorings, surfactants, humectants, lubricants and mixtures of several of these additives.

The medicament according to the invention may comprise said outer layer(s) according to a weight fraction ranging from 0% to 80% and, preferably, ranging from 10% to 50%.

Advantageously, the medicament according to the present invention is composed of a solid dosage in granule or tablet form, preferably obtained by compressing said particle agglomerate that optionally compresses said outer layer(s), or else in a solid form which contains said agglomerate in powdered form, which is packaged in an immediate container, such as a capsule, a gelatin capsule, a sachet or a vial.

Tests have shown that the medicaments according to the invention, in the form of a tablet containing 30 mg of cimicoxib, are at least 90% dissolved after 30 minutes in a medium based on around 0.1N HCl (+0.15% sodium lauryl sulfate).

Other tests have shown that the medicaments according to the invention, in the form of a tablet containing 10 mg or even 5 mg of cimicoxib, are at least 65% dissolved after 15 minutes in a medium based on 0.1N HCl.

A process for preparing a medicament according to the invention, as defined previously, comprises the following successive steps:
(i) preparing a sprayable liquid based on micronized grains of said specific (cf. formula (I) above) cyclooxygenase-2 inhibitor, especially an imidazole such as cimicoxib, which are in solution or in suspension in at least one hydrophilic polymer;
(ii) spraying said liquid, in a granulator, onto inert solid particles based on at least one excipient designed to be compatible with said inhibitor, to obtain, by wet granulation, a particle agglomerate comprising the product of spraying the solution or suspension of said grains;
(iii) optionally compressing the particle agglomerate obtained in (ii); and
(iv) optionally covering the agglomerate obtained in (ii) or in (iii) with at least one outer layer comprising compatible additives chosen from the group consisting of disintegrating agents, fillers, pigments, flavorings, surfactants, humectants, lubricants and mixtures of several of these additives.

According to one preferred feature of the invention, said granulator used in step (ii) is of the fluidized air bed type.

According to one embodiment of the invention, in order to implement this wet granulation a fluidized air bed granulator is used operating at a relative pressure approximately ranging from 1 bar to 1.5 bar, with a hot air inlet temperature in this granulator ranging from 40 to 75° C. and a temperature of the solid particles ranging from 30 to 50° C.

Preferably, the step (i) is specifically implemented by completely dissolving said inhibitor in said hydrophilic polymer(s), to obtain an improved bioavailability for the medicament according to the invention.

It should be noted that the medicaments according to the invention can be used for therapeutic and/or prophylactic treatment of diverse inflammations of a human or animal body, or for any other dysfunction of this body that is caused by cyclooxygenase-2.

The aforementioned features of the present invention, and also others, will be better understood when reading the following description of several embodiments of the invention, given by way of illustration and being nonlimiting.

### CONTROL EXAMPLE

A “control” medicament that did not conform to the invention was prepared comprising a tablet of conventional formula composed of:
a core based on a mixture of solid particles of cimicoxib and lactose;
a granulation liquid containing purified water and a surfactant based on a polysorbate according to a weight fraction of 0.40%, to agglomerate said particles; and
an outer layer formed from a mixture of a disintegrating agent, a flavoring and a lubricant composed of magnesium stearate.

More specifically, the formulation of this “control” medicament was the following, per 100 g:

| | |
|---|---|
| Active principle: cimicoxib | 8.0 g |
| Croscarmellose sodium | 5.0 g |
| Pregelatinized starch | 15.0 g |
| Polysorbate | 0.40 g |
| Lactose monohydrate | 68.10 g |
| Appetent | 3.0 g |
| Magnesium stearate | 0.5 g |

This “control” particle agglomerate was prepared by wet granulation in a high-shear granulator, and the particle agglomerate obtained was converted to a “control” tablet by the technique known to a person skilled in the art.

This “control” tablet contained 30 mg of cimicoxib.

### Example 1 According to the Invention

A first medicament according to the invention was prepared based on a particle agglomerate which comprised a substrate, based on solid particles agglomerated by the product of spraying a suspension according to the invention, and which is, in addition, provided with an outer layer.

This agglomerate was composed of a pharmaceutical composition of the following formulation (as weight fractions, outer layer excluded):

| Substrate: | |
|---|---|
| lactose particles | 72.90% |
| Liquid to be sprayed in the | |
| form of a suspension: | |
| micronized grains of cimicoxib | 8.74% |
| polyvinylpyrrolidone PVP “K25” | 13.11% |
| polyethylene glycol PEG “400” | 1.96% |
| sodium lauryl sulfate | 3.28% |

The outer layer covering the particle agglomerate had the following formulation (in grams):

| | |
|---|---|
| Disintegrating agent “acdisol” | 5 g |
| Flavoring | 3 g |
| Lubricant (magnesium stearate) | 0.5 g |

As a first step, the sprayable liquid based on cimicoxib was prepared as follows. Firstly, the hydrophilic polymer PVP was dispersed with stirring. When a clear solution was obtained, the other hydrophilic polymer PEG was added. Added slowly to the solution thus obtained was the micronized active principle (cimicoxib) that was then mixed with stirring for 30 minutes. Finally, the lauryl sulfate was added with stirring for 3 minutes.

In a second step, the suspension of cimicoxib thus obtained was sprayed in a fluidized air bed granulator over the inert heated particles of the substrate (composed of lactose), under the following conditions established for a batch of 300 g:

| | |
|---|---|
| Relative spraying pressure | 1 bar |
| Hot air inlet temperature | 60° C. |
| Air outlet temperature | 33° C. |
| Particle temperature | 34° C. |
| Spraying duration | 36 minutes. |

Next, the granule or particle agglomerate thus obtained was converted to a tablet, by covering it with the aforementioned outer layer, or else it was placed inside a capsule, by using in either case the techniques known to a person skilled in the art to obtain a suitable dosage.

In order to convert each granule to a tablet and with reference to a batch of 100 g, 91.5 g of the granule obtained was mixed with 8.5 g of the outer layer composition described above, using, for example, a mixer of the planetary mixer type or a tumble mixer.

It should be noted that this tablet may be obtained using a reciprocating or else rotary tableting machine.

### Example 2 According to the Invention

A second medicament according to the invention was prepared based on a particle agglomerate which comprised a substrate, based on solid particles agglomerated by the product of spraying another suspension according to the invention, and which is, in addition, provided with an outer layer.

This agglomerate was composed of a pharmaceutical composition of the following formulation (as weight fractions, outer layer excluded):

| Substrate: | |
|---|---|
| lactose particles | 61.97% |
| Liquid to be sprayed in the | |
| form of a suspension: | |
| micronized grains of cimicoxib | 8.74% |
| polyvinylpyrrolidone PVP “K25” | 21.86% |
| polyethylene glycol PEG “400” | 1.97% |
| sodium lauryl sulfate | 5.46% |

The outer layer covering the particle agglomerate had the same formulation as in Example 1.

The sprayable liquid, the particle agglomerate incorporating it and the tablet finally obtained from this agglomerate were prepared using the process described in Example 1, except for the hot air outlet temperature which was between 33 and 38° C. and the particle temperature which was between 45 and 50° C.

By way of indication, the solubility S0 of the cimicoxib powder alone and the solubility S2 of the particle agglomerate according to this second example of the invention were measured by the HPLC (high performance liquid chromatography) technique and in an approximately 0.1N HCl medium, with the following results (solubilities in milligrams/liter):

S0=3.1 mg/l and S2=26.8 mg/l.

This result shows that the particle agglomerate according to this second example of the invention has a greatly improved solubility in acid medium.

The tablet according to this second embodiment of the invention contained 30 mg of cimicoxib.

### Example 3 According to the Invention

A third medicament according to the invention was prepared based on a particle agglomerate which comprised a substrate, based on solid particles agglomerated by the product of spraying another suspension according to the invention, and which is, in addition, provided with an outer layer.

This agglomerate was composed of a pharmaceutical composition of the following formulation (as weight fractions, outer layer excluded):

| Substrate: | |
|---|---|
| lactose particles | 53.02% |
| citric acid | 16.39% |
| sodium lauryl sulfate | 2.73% |
| Liquid to be sprayed in the | |
| form of a suspension: | |
| micronized grains of cimicoxib | 8.74% |
| polyvinylpyrrolidone PVP “K25” | 13.11% |
| polyethylene glycol PEG “400” | 3.28% |
| sodium lauryl sulfate | 2.73% |

The outer layer covering the particle agglomerate had the same formulation as in Example 1.

The sprayable liquid, the particle agglomerate incorporating it and the tablet finally obtained from this agglomerate were prepared by using the process described in Example 1, except that:
citric acid and sodium lauryl sulfate (in the same amount as in the sprayable liquid) were incorporated into the preheated substrate; and
the hot air outlet temperature was between 33 and 38° C. and the particle temperature was between 45 and 50° C.

The tablet according to this third embodiment of the invention contained 30 mg of cimicoxib.

### Example 4 According to the Invention

A fourth medicament according to the invention was prepared based on a particle agglomerate which comprised a substrate, based on solid particles agglomerated by the product of spraying another suspension according to the invention, and which is, in addition, provided with an outer layer.

This agglomerate was composed of a pharmaceutical composition of the following formulation (as weight fractions, outer layer excluded):

| Substrate: | |
|---|---|
| lactose particles | 57.61% |
| citric acid | 16.39% |
| sodium lauryl sulfate | 2.73% |
| Liquid to be sprayed in the | |
| form of a suspension: | |
| micronized grains of cimicoxib | 4.37% |
| polyvinylpyrrolidone PVP “K25” | 13.11% |
| polyethylene glycol PEG “400” | 3.06% |
| sodium lauryl sulfate | 2.73% |

The outer layer covering the particle agglomerate had the same formulation as in Example 1.

The sprayable liquid, the particle agglomerate incorporating it and the tablet finally obtained from this agglomerate were prepared by using the exact same process as described in Example 3.

### Example 5 According to the Invention

A fifth medicament according to the invention was prepared based on a particle agglomerate which comprised a substrate, based on solid particles agglomerated by the product of spraying a solution according to the invention, but which was free of an outer layer, contrary to the Examples 1 to 4.

This agglomerate was composed of a pharmaceutical composition of the following formulation (in weight fractions):

| Substrate: | |
|---|---|
| lactose particles | 59.80% |
| Microcrystalline cellulose | 6.65% |
| Liquid to be sprayed in the | |
| form of a solution: | |
| micronized grains of cimicoxib | 3.30% |
| polyethylene glycol PEG “400” | 30.25% |

As a first step, the liquid to be sprayed was prepared as follows. Added slowly to a solution of PEG “400” were the micronized grains of the active principle (cimicoxib), that were then mixed with stirring for 20 minutes. A solution of cimicoxib in this PEG was obtained.

As a second step, the solution thus obtained was sprayed in a fluidized air bed granulator, onto the heated inert substrate particles (composed of lactose with the addition of microcrystalline cellulose), under the following conditions established for a batch of 200 g:

| | |
|---|---|
| Relative spraying pressure | 1.5 bar |
| Hot air inlet temperature | 70° C. |
| Air outlet temperature | 33° C. |
| Particle temperature | 34 to 45° C. |
| Spraying duration | 17 minutes |

By way of indication, the solubility S5 of this particle agglomerate according to this fifth example of the invention was compared to the solubility S0 of the cimicoxib powder alone by the HPLC technique and in an approximately 0.1N HCl medium, with the following results (milligrams/liter):

S0=3.1 mg/l and S5=23.9 mg/l.

This result shows that the granule or particle agglomerate according to this fifth example of the invention has a greatly improved solubility in acid medium.

This granule was then packaged directly inside a gelatin capsule.

### Example 6 According to the Invention

A sixth medicament according to the invention was prepared based on a particle agglomerate which comprised a substrate, based on solid particles agglomerated by the product of spraying a solution according to the invention, but which was free of an outer layer, contrary to the Examples 1 to 4.

This agglomerate was composed of a pharmaceutical composition of the following formulation (in weight fractions):

| Substrate: | |
|---|---|
| lactose particles | 62.55% |
| Microcrystalline cellulose | 6.95% |
| Liquid to be sprayed in the | |
| form of a solution: | |
| micronized grains of cimicoxib | 3.00% |
| polyethylene glycol PEG “400” | 27.50% |

This particle agglomerate was obtained by implementing the exact same process described above in Example 5.

This granule was then packaged directly inside a gelatin capsule.

### Measurements of the Bioavailability of the “Control” Medicament and of Several Medicaments According to the Invention

Administered orally to four dogs (two males and two females), all of the “Beagle” breed, were: the “control” tablet, the second and third tablets according to the invention and the gelatin capsule according to the fifth example of the invention, respectively obtained in the Examples “control”, 2, 3 and 5 above. Each dog thus received the same dose of 30 mg of cimicoxib during the ingestion of these four types of formulations, while spacing each administration over a minimum time interval of 6 days.

Blood samples relating to each tablet or gelatin capsule administered to each of the four dogs were collected, at various times following each administration of these pharmaceutical forms, in order to carry out an analysis of the bioavailability of these products in terms of concentration Cₘₐₓ (plasma level of cimicoxib, in μg/ml) and of the area under the curve (AUC in ∥g·h/ml, calculated over 10 hours). These collection times (expressed in hours) were the following:
0; 0.25 h; 0.5 h; 0.75 h; 1 h; 1.5 h; 2 h; 3 h; 4 h; 5 h; 6 h; 8 h; 10 h; 24 h; 32 h; 48 h.

Table 1 below gives the average results obtained for the “control” tablet, the second and third tablets according to the invention and the gelatin capsule according to the fifth example of the invention administered to all four dogs.

TABLE 1

| | | Cₘₐₓ | “AUC” (μg · h/ml, |
|---|---|---|---|
| Tablets tested | Tₘₐₓ (h) | (μg/ml) | over 10 h) |
| “Control” tablet | 2.13 | 0.2825 | 1.341 |
| Second tablet of | 2.76 | 0.6279 | 2.606 |
| the invention | | | |
| Third tablet of the | 2.00 | 0.6215 | 2.355 |
| invention | | | |
| Gelatin capsule | 1.33 | 1.648 | 5.938 |
| according to the | | | |
| fifth example of | | | |
| the invention | | | |

This table shows that the medicaments according to the invention have an assimilation in the body (i.e. a bioavailability) that is greatly improved relative to that of the “control” tablet, as is shown by the higher values of the concentration Cₘₐₓ and of the “AUC” area.

This table also shows that spraying a cyclooxygenase-2 inhibitor specifically in the form of a solution (i.e. dissolved in the hydrophilic polymer(s)) on the solid particles of the inert substrate further improves the bioavailability of the medicaments according to the invention by oral means.

1. A medicament that is intended for oral administration and that has an improved bioavailability, said medicament comprising an agglomerate based on inert solid particles that are based on at least one excipient, said agglomerate comprising a cyclooxygenase-2 inhibitor and at least one hydrophilic polymer, characterized in that said agglomerate comprises the product of spraying said particles with a solution or suspension of micronized grains of said inhibitor in said polymer(s) in order to agglomerate said particles, and in that said inhibitor is composed of at least one compound of formula (I) or a salt or solvate of said compound: 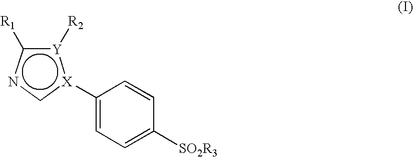
where:
one of the components X and Y represents N and the other represents C;
R₁ represents a hydrogen, methyl, halogen, cyano, nitro, —CHO, —COCH₃ or —COOR₄ group;
R₂ represents an aryl or heteroaryl group optionally substituted by one or more groups chosen independently from halogen, C₁₋₈ alkyl, C₁₋₈ haloalkyl, R₄OC₀₋₈ alkyl, R₄SC₀₋₈ alkyl, cyano, nitro, —NR₄R₆, —NR₄SO₂R₅, —SOR₅, —SO₂R₅, —SO₂NR₄R₆, or —CONR₄R₆ groups;
R₃ represents a C₁₋₈ alkyl, C₁₋₈ haloalkyl or —NR₄R₆ group;
R₄ represents a hydrogen, C₁₋₈ alkyl or C₀₋₈ alkyl aryl group (where the aryl group may optionally be substituted by one or more groups chosen from C₁₋₈ alkyl, halogen, C₁₋₈ haloalkyl, cyano, nitro, R₇OC₀₋₈ alkyl, R₇SC₀₋₈ alkyl, —NR₇R₈, —NR₇COR₅, —COR₇ or —COOR₇ groups);
R₅ represents a C₁₋₈ alkyl or C₁₋₈ haloalkyl group;
R₆ represents a hydrogen, C₁₋₈ alkyl, aryl C₁₋₈ alkyl (where the aryl group may optionally be substituted by one or more groups chosen from C₁₋₈ alkyl, halogen, C₁₋₈ haloalkyl, cyano, nitro, R₇OC₀₋₈ alkyl, R₇SC₀₋₈ alkyl, —NR₇R₈, —NR₇COR₅, —COR₇ or —COOR₇ groups), —COR₈ or —COOR₈ group;
R₇ represents a hydrogen, C₁₋₈ alkyl or benzyl group; and
R₈ represents a C₁₋₈ alkyl or C₁₋₈ haloalkyl group;
wherein the aryl group in the definitions above represents a phenyl or naphthyl group; and
the heteroaryl group in the definitions above represents a pyridine, pyrazine, pyrimidine or pyridazine group, which may optionally be fused to a benzene ring.

2. The medicament as claimed in claim 1, characterized in that said inhibitor is composed of at least one imidazole.

3. The medicament as claimed in claim 1, characterized in that said agglomerate is capable of being obtained by wet granulation in a fluidized air bed device.

4. The medicament as claimed in claim 1, characterized in that said agglomerate comprises the product of spraying a solution of said inhibitor in said polymer(s).

5. The medicament as claimed in claim 1, characterized in that said particles of excipient(s) are soluble or dispersible in an aqueous medium.

6. The medicament as claimed in claim 1, characterized in that said agglomerate comprises said inhibitor according to a weight fraction ranging from 1% to 20%.

7. The medicament as claimed in claim 6, characterized in that said agglomerate comprises said inhibitor according to a weight fraction ranging from 3% to 10%.

8. The medicament as claimed in claim 6, characterized in that said agglomerate comprises said excipient(s) according to a weight fraction ranging from 10% to 80%.

9. The medicament as claimed in claim 8, characterized in that said agglomerate comprises said excipient(s) according to a weight fraction ranging from 30% to 75%.

10. The medicament as claimed in claim 6, characterized in that said agglomerate comprises said hydrophilic polymer(s) according to a weight fraction ranging from 3% to 30%.

11. The medicament as claimed in claim 10, characterized in that said agglomerate comprises said hydrophilic polymer(s) according to a weight fraction ranging from 12% to 25%.

12. The medicament as claimed in claim 1, characterized in that said or at least one of said hydrophilic polymer(s) is chosen from the group consisting of polyvinylpyrrolidones, polyethylene glycols or macrogols, polyvinyl alcohols, cellulose polymers selected from the group consisting of hydroxypropylmethyl cellulose, hydroxypropyl cellulose and carboxymethyl cellulose, methacrylic copolymers, starch, dextrins, gelatin and blends of several of these polymers.

13. The medicament as claimed in claim 12, characterized in that said or at least one of said hydrophilic polymer(s) is chosen from the group consisting of polyvinylpyrrolidones and polyethylene glycols or macrogols.

14. The medicament as claimed in claim 13, characterized in that said or at least one of said polyethylene glycol(s) or macrogol(s) has a weight-average molecular weight M_{w} ranging from 190 to 9000 g/mol.

15. The medicament as claimed in claim 14, characterized in that said or at least one of said polyethylene glycol(s) or macrogol(s) has a weight-average molecular weight M_{w} ranging from 250 to 600 g/mol.

16. The medicament as claimed in claim 13, characterized in that said hydrophilic polymers comprise a blend of said polyethylene glycol or macrogol and a polyvinylpyrrolidone having a weight-average molecular weight M_{w} ranging from 2000 to 1 000 000 g/mol.

17. The medicament as claimed in claim 16, characterized in that said polyvinylpyrrolidone has a weight-average molecular weight M_{w} ranging from 20 000 to 55 000 g/mol.

18. The medicament as claimed in claim 1, characterized in that said product of spraying the solution or suspension of said inhibitor in said polymer(s) comprises, in addition, at least one amphoteric, ionic or nonionic surfactant, the weight fraction of said surfactant(s) in said agglomerate ranging from 0.1% to 6%.

19. The medicament as claimed in claim 18, characterized in that said surfactant is sodium lauryl sulfate.

20. The medicament as claimed in claim 1, characterized in that said excipient(s) comprises or comprise water-soluble or water-dispersible inert particles which are chosen from the group consisting of sugars, preferably lactose or saccharose, starch hydrolysates such as maltodextrin, microcrystalline cellulose, sorbitols and mixtures of several of these compounds.

21. The medicament as claimed in claim 1, characterized in that said agglomerate comprises, in addition, at least one acid that is mixed with said particles of excipient(s), wherein said acid is chosen from citric acid, tartaric acid or fumaric acid.

22. The medicament as claimed in claim 1, characterized in that it comprises at least one outer layer covering said agglomerate and comprising compatible additives chosen from the group consisting of disintegrating agents, fillers, pigments, flavorings, surfactants, humectants, lubricants and mixtures of several of these additives.

23. The medicament as claimed in claim 1, characterized in that it is composed of said agglomerate of solid particles being in the form of a powder packaged in an immediate container, or in the form of a tablet.

24. A process for preparing a medicament as claimed in claim 1, characterized in that it comprises the following successive steps:
(i) preparing a sprayable liquid based on micronized grains of said cyclooxygenase-2 inhibitor which are in solution or in suspension in at least one hydrophilic polymer;
(ii) spraying said liquid, in a granulator, onto inert solid particles based on at least one excipient designed to be compatible with said inhibitor, to obtain, by wet granulation, a particle agglomerate comprising the product of spraying the solution or suspension of said grains;
(iii) optionally compressing the particle agglomerate obtained in (ii); and
(iv) optionally covering the agglomerate obtained in (ii) or in (iii) with at least one outer layer comprising compatible additives chosen from the group consisting of disintegrating agents, fillers, pigments, flavorings, surfactants, humectants, lubricants and mixtures of several of these additives.

25. The process as claimed in claim 24, characterized in that said granulator is of the fluidized air bed type.

26. The process as claimed in claim 24, characterized in that the hot air inlet temperature in said granulator is between 40° C. and 75° C.

27. The process as claimed in claim 24, characterized in that the temperature of said solid particles in said granulator is between 30° C. and 50° C.

28. The process as claimed in claim 24, characterized in that the step (i) is implemented by completely dissolving said inhibitor in said polymer(s).
